(19) United States
(12) Patent Application Publication
(10) Pub. No.: US 2006/0292188 A1
(43) Pub. Date: Dec. 28, 2006

(54) Ophthalmic solution with a flavoring agent

(76) Inventors: Joseph C. Salamone, Boca Raton, FL (US); Erning Xia, Penfield, NY (US)

Correspondence Address: Bausch & Lomb Incorporated, One Bausch & Lomb Place, Rochester, NY, 14604-2701, US

(21) Appl. No.: 11/431,949

(22) Filed: May 11, 2006

Related U.S. Application Data
(60) Provisional application No. 60/687,085, filed on Jun. 3, 2005.
(60) Provisional application No. 60/704,567, filed on Aug. 2, 2005.

(51) Int. Cl. A61K 9/00

(52) U.S. Cl. 424/400

(57) ABSTRACT

The invention provides an ophthalmic solution with at least one flavoring agent to mask flavors in the solution or add flavor to the solution. The flavoring agent can be a sweet flavoring agent (sweetener), or combinations of a sweetener with a sour flavoring agent or a bitter flavoring agent or mixtures thereof. The flavoring agent may also be natural flavors, natural fruit flavors, artificial flavors, artificial fruit flavors, flavor enhancers and mixtures thereof, which may be combined with a sweetener, a sour flavoring agent, a bitter flavoring agent, and mixtures thereof. The invention may be utilized as a contact lens solution, an eye drop formulation, and a pharmaceutical composition containing at least one active pharmaceutical ingredient for the treatment of eye diseases.

### CROSS REFERENCE

This application claims the benefit of Provisional Patent Application No. 60/687,085 filed Jun. 3, 2005 and Provisional Patent Application No. 60/704,567 filed Aug. 2, 2005 and are incorporated herein by reference.

### FIELD OF INVENTION

This invention relates to an ophthalmic solution with at least one flavoring agent to mask flavor of the solution or to add flavor to the solution.

### BACKGROUND

Millions of people wear contact lenses. People wear contact lenses for a variety reasons. Contact lenses can move with your eye, allow a natural field of view, have no frames to obstruct your vision and greatly reduce distortions. Contact lenses do not fog up, like glasses, nor do they get splattered by mud or rain. Many people feel they look better in contact lenses. There are many types of contact lenses available today. However, contact lenses require careful cleaning to disinfect the lens for any bacteria, viruses, or fungi.

Contact lenses generally fall into two categories. The hard or rigid corneal type lenses are formed from materials prepared by the polymerization of sioloxanyl acrylates-3-methacryloyloxypropyltris(trimethylsiloxyl)silane. The hydrogel or soft type of lenses are made by polymerizing such monomers as 2-hydroxyethyl methacrylate (HEMA) or, in the case of extended wear lenses, made by polymerizing silicon-containing monomers or macromonomers. Solutions that wet the lenses before insertion in the eye are required for both the hard and soft contact lenses, although their formulations tend to differ based on differences in the lens properties. After the contact lenses are inserted in the eye, ophthalmic solutions for rewetting, lubricating, and/or enhancing wearer comfort are sometimes applied to the eye by means of a drop dispenser.

Ophthalmic solutions include a wide variety of aqueous formulations for an eye and a contact lens care as well as many therapeutic treatments. Isotonic solutions for improving the comfort of wearing soft contact lenses by being added directly to the contact lens in the eye are well known. Such solutions typically contain viscosity enhancing agents, lubricants, surfactants, buffers, preservatives, and salts. Some eye drops use menthol or camphor as a cooling agent for a cool sensation in the eyes to relieve minor eye irritations. Present in very minor amounts, these cooling agents are do not flavor the ophthalmic solution. Examples are Rohto V For Eyes, Lubricant/Redness Reliever Eye Drops (inactive ingredient: menthol) and Rohto Zi For Eyes (Povidone) Lubricant Eye Drops (inactive ingredient: camphor).

Various multipurpose lens care solutions have been developed over the years to ensure that contact lenses are essentially pathogen and deposit free. These contact lens solutions typically include anti-microbial substances as well as cleaning (active against both lipids and proteins), wetting, conditioning, and other agents for the disinfection and cleaning of contact lenses during storage after wear. So-called, multipurpose solutions (MPS) can disinfect and clean without harming the eye or lens in addition to wetting.

In-eye ophthalmic solutions are generally administered by means of a plastic bottle with an attached dropper or snap cap. The maximum volume of a solution that can be added into the lower eyelid sack is generally 30 μl. An excess of the solution administered is eliminated via nasal drainage, which eventually flows to the mouth.

The unpleasant bitter taste of ophthalmic solutions has been a major complaint from patients and contact lens wearers. This invention provides the palatable ophthalmic solutions with a variety of flavors to mask unpleasant taste or add flavor to improve the taste.

### SUMMARY OF INVENTION

The invention provides an ophthalmic solution with at least one flavoring agent to mask flavors in the solution or to add flavor to the solution. The flavoring agent can be a sweet flavoring agent (sweetener), which can be combined with a sour flavoring agent, a bitter flavoring agent, or mixtures thereof. The invention may be utilized as a contact lens solution, an eye drop formulation, and a pharmaceutical composition containing at least one active pharmaceutical ingredient for the treatment of an eye disease.

The invention provides an ophthalmic solution with a sweetener, where the sweetener is a natural sugar or a sugar substitute of artificial origin. The invention also provides an ophthalmic solution with a flavoring agent selected from natural flavors, natural fruit flavors, artificial flavors, artificial fruit flavors, flavor enhancers, and mixtures thereof. These flavoring agents can be used in combination with a sweetener, a sour flavoring agent, a bitter flavoring agent, or mixtures thereof.

### BRIEF DESCRIPTION OF THE DRAWINGS

FIG. 1 illustrates the portions of the human mouth and throat which respond to different tastes.

### DETAILED DESCRIPTION OF THE INVENTION

Generally speaking, an ophthalmic solution may have its own flavor or not have any flavor. In the ophthalmic solution of the invention, a flavoring agent is used to mask flavor in the solution or add flavor to the solution. The flavoring agent may be selected from four major tastes: sourness, sweetness, saltiness, and bitterness, or combination thereof. Preferably, the flavoring agent has sweetness by a sweet flavoring agent (a sweetener), a combination of a sweetener with other flavoring agents, or mixtures thereof. Most preferably, the flavoring agent may be a sweetener, a combination of a sweetener with a sour flavoring agent, a combination of a sweetener and a bitter flavoring agent, or mixtures thereof. In addition, the flavoring agents can be selected from the group consisting of natural flavors, natural fruit flavors, artificial flavors, artificial fruit flavors, flavor enhancers and mixtures thereof.

The ophthalmic solutions formulated according to the invention are physiologically compatible. Specifically, the solution must be “ophthalmically safe” for use in the eye or with a contact lens, meaning that a contact lens treated with the solution is generally suitable and safe for direct placement on the eye without rinsing. In other words, for a contact lens solution, the solution is safe and comfortable for daily contact with the eye via a contact lens that has been wetted with the solution. An ophthalmically safe solution has a tonicity and pH that is compatible with the eye and comprises materials, and amounts thereof, that are non-cytotoxic according to ISO standards and U.S. FDA (Food & Drug Administration) regulations. The solution should be sterile in that the absence of microbial contaminants in the product prior to release must be statistically demonstrated to the degree necessary for such products.

This invention is directed to an ophthalmic solution with at least one flavoring agent to mask flavor in the solution or to add flavor to the solution. The flavoring agent may be added to an already prepared ophthalmic solution or be added during the preparation of the ophthalmic solution using formulation techniques known in the art. In general, this requires only simple mixing. The flavoring agent is suitably present in an amount to cause taste, for example, from about 0.0001 to 20 weight percent. Preferably, the flavoring agent may be present in an amount ranging from 0.01 to 10 weight percent, most preferably in an amount from 0.1 to 5 weight percent.

According to the invention, the flavoring agent may be formulated into a contact lens solution, an eye drop formulation, and a pharmaceutical composition containing at least one active pharmaceutical ingredient for the treatment of an eye disease.

In one aspect, the flavoring agents may be formulated into contact lens care solutions, such as wetting solutions, soaking solutions, cleaning solution, conditioning solutions, disinfecting solution and storing solutions for daily or weekly uses. Wetting solutions sooth and comfort the eyes by providing lubrication, when lenses becomes dry and uncomfortable. The solutions can be used for rinsing after daily cleaning, as a final rinse after disinfection, and prior to lens insertion. The solutions may also be used to store your lenses during disinfection, cleaning, wetting, and conditioning.

The invention can be used with all contact lens solutions and all types of contact lenses such as conventional rigid gas permeable lenses, traditional hydrogels, and silicone hydrogels. Traditional hydrogels are commonly prepared from monomers such as 2-hydroxyethyl methacrylate with suitable crosslinking agents, N-vinylpyrrolidone, glycerol methacrylate, methacrylic acid or acid esters, and the like. Such lenses absorb significant amounts of water, which amounts range from about 4 to about 80 percent by weight. Rigid gas permeable lenses and silicone hydrogels are formulated with siloxanyl monomers and macromonomers with suitable wetting hydrophilic monomers.

In another aspect, the invention may be formulated as an eye drop to soothe eye irritation, a moisturizing solution, a contact lens rewetting solution, and a contact lens lubricating solution. Examples of eye drops would be topical artificial tears and lubricants for dry eye which are available at Over-The-Counter (OTC) without prescription. These tear substitutes increase humidity at the ocular surface and to improve lubrication. In addition, artificial tears smooth the corneal surface of dry eye patients, an effect that contributes to improved vision.

According to the invention, the flavoring agent may also be formulated into pharmaceutical composition which contains an active pharmaceutical ingredient for the treatment of eye disease. The ophthalmic solution, which may be available with prescription or OTC, can relieve itchy, red, teary, and irritated eyes associated with seasonal allergies. Examples can be an ophthalmic solution to reduce both internal and external inflammation of the eye, to treat ocular hypertension and glaucoma with an ophthalmic beta blocker, or to supply a high potency antioxidant and mineral supplement to the eyes.

Flavor is the sensation caused by those properties of any substance taken into the mouth which stimulates one or both of the senses of taste and smell and/or also the general pain, tactile, and temperature receptors in the mouth. There are four major tastes: (1) sourness, (2) sweetness, (3) saltiness, and (4) bitterness. Sourness is the simplest taste. Usually, the more hydrogen ions, the sourer the solution becomes. The most common taste activators for sweetness are sugars, although there are many other compounds that have sweet taste. Sodium chloride has the most pure salty taste. The examples of the compounds for bitterness are caffeine, nicotine, quinine and brucine.

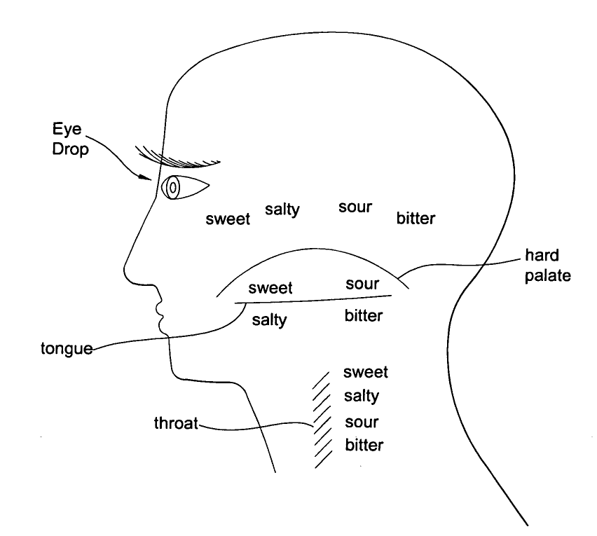

FIG. 1 depicts the portions of the human mouth and throat which respond to different tastes. Taste, or gustation, is the combined impression we receive when free nerve endings and taste buds in the mouth detect various stimuli. The free nerve endings possess no receptors, but are responsible for the perception of sensations such as pain, temperature, pungency, and astringency. The taste buds are clusters of approximately 100 taste cells that occur as protuberances, called papillae, on the tongue. Taste cells lie within taste buds, which are located in various tongue papillae, hard and soft palate and root of the tongue. The mechanism of flavor perception is not well understood, but it is believed that the arrival of a chemical stimulant on the surface of a receptor temporarily modifies the cell wall and produces an electrochemical impulse. This impulse is then transmitted through a nerve cell to the brain, where it is decoded into sensory information in the cerebral cortex. Taste depends mainly on the contact of soluble matter with the terminal organs (connected with branches of the glossopharyngeal and other nerves) in the papillae on the surface of the tongue. The four basic tastes (sweet, salt, sour, and bitter) are unevenly distributed on the tongue, as seen in FIG. 1. The base of the tongue is considered most sensitive to bitter substances, the point to sweet and acid substances.

Flavoring agents may be a single chemical or a blend of chemicals whose primary purpose is to provide all or part of the particular flavor or effect to any products such as ophthalmic products. The flavoring agent can be in the forms of oils or extracts. The flavoring agent may be acidic, basic, neutral or salt.

The flavoring agent may be a sweetener which may be preferably used to mask the inherent flavors of the ophthalmic solution. Sweeteners are the food additives of natural sugar, or sugar substitutes of artificial origin. The invention provides an ophthalmic solution with a flavoring agent, wherein the flavoring agent is a sweetener.

The sweetener used may be selected from a wide range of materials including water-soluble sweeteners, water-soluble artificial sweeteners, water-soluble sweeteners derived from naturally occurring water-soluble sweeteners, and mixtures thereof. Without being limited to particular sweeteners, representative categories and examples are shown in Table 1.

TABLE 1

| Water-soluble Sweeteners | | Water-soluble Sweeteners |
|---|---|---|
| (monosaccharides, | | derived from naturally |
| disaccharides and | Water-soluble | occurring Water-soluble |
| polysaccharides) | Artificial Sweeteners | Sweeteners |
| xylose, ribulose, glucose | soluble saccharin salts, i.e., | chlorinated derivatives of |
| (dextrose), mannose, | sodium or calcium | ordinary sugar (sucrose), |
| galactose, fructose | saccharin salts, cyclamate | known, for example under |
| (levulose), sucrose (table | salts, the sodium, | the produce designation of |
| sugar), maltose, invert sugar | ammonium or calcium salt | sucralose. |
| (a mixture of fructose and | of 3,4-dihydro-6-methyl- | |
| glucose derived from | 1,2,3-oxathiazine-4-one- | |
| sucrose), partially | 2,2-dioxide, the potassium | |
| hydrolyzed starch, corn | salt of 3,4-dihydro-6- | |
| syrup solids, | methyl-1,2,3-oxathiazine-4- | |
| dihydrochalcones, monellin, | one2,2-dioxide | |
| steviosides, glycyrrhizin, | (Acesulfame-K). | |
| and sugar alcohols such as | | |
| sorbitol, mannitol, xylitol, | | |
| maltitol, hydrogenated | | |
| starch hydrolysates and | | |
| mixtures thereof. | | |

Preferred sugar-based sweeteners in the invention are dextrose, sucrose, and fructose, sorbitol, mannitol, xylitol and mixtures thereof.

The use of artificial sugar products as the flavoring agent provides an ophthalmic solution for patients concerned with diet management. Artificial/synthetic sweeteners, sugar alternatives, alternative sweeteners, non-nutritive sweeteners, non-caloric/low-cal/low-carb sweeteners, diabetic-safe sweeteners are all interchangeable and synonymous for the purposes of the invention. There are currently five low-calorie sweeteners approved by the Food and Drug Administration (FDA), including acesulfame potassium, aspartame, neotame, saccharin and sucralose. These sweeteners are hundreds of times sweeter than sucrose and do not contribute calories to the diet. Sucralose, chemically known as 1,6-dichloro-1,6-dideoxy-β-D-fructofuranosyl-4-chloro-4-deoxy-α-D-galactopyranoside, is a non-nutritive, high-intensity sweetener made from a process that begins with sucrose and sold under the Splenda® trademark. The chemical structures of sugar and sucralose are: 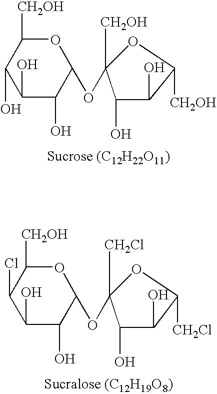
Sucralose contains covalently bound chlorine atoms, which create a sweetener that is 600 times sweeter than sugar.

There are also a number of reduced-calorie sweeteners (polyols) available in the U.S., including erythritol, hydrogenated starch hydrosylates, isomalt, lactitol, maltitol, mannitol, sorbitol and xylitol. Polyols contribute between and 0.2 and three calories per gram as opposed to sucrose, which contributes four calories per gram. Polyols not only contribute sweetness but also bulk, and are used in a variety of products.

Sugarless sweeteners in the invention may include, but are not limited to, are sucralose, isomalt, aspartame, saccharin, lacitol, and other sweet replacers.

As discussed above, any flavoring agent or combination of flavoring agent may be used in the ophthalmic solution of the invention. Without being limited to particular flavors, examples of flavoring agents are shown in Table 2 (available from International Flavors & Fragrances, Inc.

(http://www.iff.com/_{—}85256C33004F6FEB.NSF/FlavIngredients!OpenForm).

TABLE 2
Examples of Flavoring Agents

| | |
|---|---|
| 6-Methyl Coumarin | Artificial & Kosher |
| Anethole USP | |
| Cassia Oil | Natural & Kosher |
| Cassia Oil Redistilled | Natural & Kosher |
| Cinnamon Bark Oil | Natural & Kosher |
| Clove Bud Oil English Distilled SAS | Natural & Kosher |
| Clove Leaf Oil Redistilled | Natural & Kosher |
| Cocoa Distillate (Nat.) | Natural & Kosher |
| Cocoa Essence Dark | Natural & Kosher |
| Cocoa Essence White | Natural & Kosher |
| Coriander Oil | Natural & Kosher |
| delta Decalactone | Natural & Kosher Parve |
| Dimethyl Benzyl Carbinyl Butyrate | Artificial & Kosher Parve |
| Ethyl-2-Methyl Butyrate | Natural & Kosher Parve |
| Ethyl-3-Hydroxy Butyrate | Artificial & Kosher Parve |
| Ethyl Butyrate | Natural & Kosher Parve |
| Ethyl Iso Butyrate | Natural & Kosher Parve |
| Ethyl Iso Valerate | Natural & Kosher Parve |
| Ethyl Oxanoate 369 | Artificial & Kosher Parve |
| Eucalyptus Oil 80% | Natural & Kosher |
| Farnesene 1% PG/ETOH | Artificial & Kosher Parve |
| Furfurrole 302 | Artificial & Kosher Parve |
| γ-Decalactone | Natural & Kosher Parve |
| γ-Hexalactone | Natural & Kosher Parve |
| γ-Octalactone | Natural & Kosher Parve |
| γ-Dodecalactone | Natural & Kosher Parve |
| Ginger Oil Chinese | Natural & Kosher |
| Ginger Oil Nigerian English Distilled SAS | Natural & Kosher |
| Grapefruit Key | Natural WONF & Kosher |
| | Parve |
| Heptan-2-One (Nat.) | Natural & Kosher |
| Hexene-3-One-4 | Artificial & Kosher Parve |
| Hexyl Acetate | Natural & Kosher Parve |
| Homo Cyclocitral, beta | Artificial & Kosher Parve |
| Honey Distillate Nat. | Natural & Kosher |
| Ionone Beta | Natural & Kosher Parve |
| Iso Amyl Iso Valerate | Natural & Kosher Parve |
| Iso Butyl Caproate | Natural & Kosher Parve |
| Iso Butyl Furyl Propionate | Artificial & Kosher Parve |
| Iso Fragarone-030 | Artificial & Kosher Parve |
| Iso Fragarone, 1% ETOH ™ | Artificial & Kosher Parve |
| Juniperberry Oil English Distilled SAS | Natural & Kosher |
| Ketone Mix | Natural & Kosher Parve |
| Kumarone ™ | Artificial & Kosher Parve |
| Lemon Oil 5X Sas | Natural & Kosher |
| Lemon Oil Terpeneless Sas | Natural & Kosher |
| Lemonless Lemon Key | Natural & Kosher Parve |
| Lime Oil Terpeneless | Natural & Kosher |
| Linalool 75/80% Ex Orange (Nat.) | Natural & Kosher |
| Linalyl Acetate (Nat.) | Natural & Kosher |
| Mangone 5% ETOH ™ | Natural & Kosher Parve |
| Methional | Natural & Non-Kosher |
| Methyl Butyric Acid (2) | Natural & Kosher Parve |
| Methyl Ketones (Nat.) | Natural & Kosher |
| Methyl Oxycyclosulfide 719 | Artificial & Kosher Parve |
| Natural Flavor (99% Vanillin) | Natural & Kosher Parve |
| Nat. Cocoa Butter Distillate | Natural |
| Nonan-2-One (Nat.) | Natural & Kosher |
| Octanal 35% (Nat.) | Natural & Kosher |
| Octen-4-one-2 | Artificial & Kosher Parve |
| Olibanum Oil English Distilled SAS | Natural & Kosher |
| Orange Oil 15X Decolorized M3706 | Natural & Kosher |
| Orange Oil 950 (10X) | Natural & Kosher |
| Orange Oil Terpeneless 2501 | Natural & Kosher |
| Oxaromate-884 | Artificial & Kosher Parve |
| Oxycyclothione-030 | Artificial & Kosher Parve |
| Paradiff ™ 0.01% ETOHGR | Natural & Kosher Parve |
| Paradiff ™ 0.01% Grapefruit Oil | Natural & Kosher Parve |
| Peach Flavor Key | Natural & Kosher Parve |
| Peppermint Oil Redistilled Yakima | Natural & Kosher |
| Peppermint Oil Spec. Fractions | Parve |
| Phenyl Ethyl 2-Methyl Butyrate | Natural & Kosher Parve |
| Phenyl Ethyl Acetate | Natural & Kosher Parve |
| Phenyl Ethyl Alcohol | Natural & Kosher Parve |
| Phenyl Oxaromate-681 | Artificial & Kosher Parve |
| Pimento Berry Oil English Distilled SAS | Natural & Kosher |
| Pimento Leaf Oil | Natural & Kosher |
| Pimento Leaf Oil Cleaned | Natural & Kosher |
| Pineapple Compound 15% ETOH GR | Natural & Kosher Parve |
| Pineapple Compound 15% PG | Natural & Kosher Parve |
| Popcorn Chemical | Artificial & Kosher Parve |
| Propionic Acid | Natural & Kosher Parve |
| Raspberry Flavor Key | Natural & Kosher Parve |
| Robustone 1.0% ETOH ™ | Natural & Kosher Parve |
| Robustone ™ | Artificial & Kosher Parve |
| Schinus Molle Oil | Natural & Kosher |
| Sclareolide | Natural & Kosher parve |
| Sesame Distillate Nat. | Natural & Kosher |
| Sinensals (Nat.) | Natural & Kosher |
| Starter Distillate 15X W/S | Natural & Kosher Dairy |
| Strawberriff | Artificial & Kosher Parve |
| Strawberry Base | Natural & Kosher Parve |
| Strawberry Flavor Key | Natural & Kosher Parve |
| Succinic Acid | Natural & Kosher Parve |
| Sulfurome-015 | Artificial & Kosher Parve |
| Sweetness Modifier | Natural & Kosher Parve |
| Tetrahydro Terrazine-014 ™ | Artificial & Kosher Parve |
| Thionol-935 | Artificial & Kosher parve |
| Thionol-966 | Artificial & Kosher Parve |
| trans-2-Hexenal | Natural & Kosher Parve |
| Trimenal Acetate 399 1% ETOH ™ | Artificial & Kosher Parve |
| Tropical Fruit Key Base | Natural & Kosher Parve |
| Undecan-2-One (Nat.) | Natural & Kosher |
| Varamol-106 10% ETOH | Artificial & Kosher Parve |
| Varamol-106 10% NEBM5 | Artificial & Kosher Parve |
| Varamol-106 10% PG | Artificial & Kosher Parve |

The following examples demonstrate the solutions of the present invention. However, it is to be understood that these examples are for illustrative purposes only and do not purport to be wholly definitive as to conditions and scope.

### EXAMPLES

In the examples below, certain chemical ingredients are identified by the following abbreviations.
HPMC: Hydroxylpropylmethylcellulose
EDTA: EthylenediamineTetraacetic Acid
BAK: Benzalkonium Chloride, commercially available from Sigma Corp.
PHMB: Poly(hexamethylene biguanide)
Dequest® 2016: Tetrasodium phosphate, (1-hydroxyethylidene)diphosphonic acid, sodium salt, available from Monsanto Co.
Tetronic® 1107: poloxamine surfactant, a tetrafunctional block copolymer surfactant, commercially available from BASF
Pluronic® P123: poloxamine surfactant, a difunctional block copolymer surfactant, commercially available from BASF
Polymer JR®: cationic polysaccharide, polyquatemium-10
Alexidine 2HCl: quaternary ammonium salts. 1,1′-Hexamethylene-bis[5-(2-ethylhexyl)biguanide]

### Example 1

### Opcon-A® Eye Drops with a Sweetener

An ophthalmic solution of Opcon-A® eye drops with sucralose was prepared with the following formulation shown below in Table 3. Opcon-A® Itching and Redness Reliever Eye Drops combine an antihistamine for itch relief with a redness reliever. Available without a prescription, Opcon-A® eye drops relieve the itching and redness caused by pollen, ragweed, grass, animal hair, and dander. The flavoring agent, sucralose, was added by mixing the indicated amount with prepared Opcon-A® eye drops.

TABLE 3
Ophthalmic Solution of Opcon-A ® Eye Drops with Sucralose

| Ingredient | % w/w |
|---|---|
| Naphazoline HCl | 0.027 |
| Pheniramine maleate | 0.315 |
| HPMC | 0.500 |
| EDTA | 0.100 |
| BAK | 0.010 |
| Boric acid | 0.800 |
| Sucralose | 2.000 |
| Sodium borate | 0.100 |
| Sodium chloride | 0.300 |

### Example 2

### Flavored Opcon-A® Eye Drop Formulation

An ophthalmic solution for Opcon-A® eye drop formulation with orange flavor was prepared with the following formulation shown below in Table 4. The orange flavoring agent was added by mixing the indicated amount with prepared Opcon-A® eye drops.

TABLE 4
Ophthalmic Solution for Opcon-A ® Eye Drop Formulation with
Orange Flavor

| Ingredient | % w/w |
|---|---|
| Naphazoline HCl | 0.027 |
| Pheniramine maleate | 0.315 |
| HPMC | 0.500 |
| EDTA | 0.100 |
| BAK | 0.010 |
| Citric Acid | 0.300 |
| Sucralose | 2.000 |
| Orange Flavor | 0.400 |

### Example 3

### Pharmaceutical Composition (Brimonidine Tartrate) with a Sweetener

An ophthalmic solution of pharmaceutical composition for glaucoma with sucralose was prepared with the following formulation shown below in Table 5. Brimonidine acts on receptors (alpha-receptors) in the blood vessels of the eye causing them to constrict. These blood vessels control the production of the watery fluid that fills the rear of the eye. When the blood vessels constrict, there is a decrease in the production of this watery fluid. Brimonidine is used in the treatment of glaucoma. This is a condition where the fluid drainage from the eye is impaired, resulting in fluid build-up and increased pressure in the eye. Sucralose was added by mixing the indicated amount with prepared Brimonidine Tartrate.

TABLE 5
Pharmaceutical Composition (Brimonidine Tartrate) with a Sucralose

| Ingredient | % w/w |
|---|---|
| Brimonidine Tartrate | 0.200 |
| BAK | 0.050 |
| Sucralose | 2.000 |

### Example 4

### Flavored Pharmaceutical Composition (Brimonidine Tartrate)

An ophthalmic solution of pharmaceutical composition for glaucoma with lemon flavor was prepared with the following formulation shown below in Table 6. Lemon flavor was added by mixing the indicated amount with prepared Brimonidine Tartrate.

TABLE 6
Pharmaceutical Composition for Glaucoma with Lemon Flavor

| Ingredient | % w/w |
|---|---|
| Brimonidine Tartrate | 0.200 |
| BAK | 0.050 |
| Citric Acid | 0.300 |
| Sucralose | 2.000 |
| Lemon Flavor | 0.400 |

### Example 5

### Multi-Purpose Solution for Contact Lenses with a Sweetener

A multi-purpose solution for contact lenses with sucralose was prepared with the following formulation shown below in Table 7. Sucralose was added by mixing the indicated amount with prepared Multi-purpose solution for contact lenses.

TABLE 7
Multi-Purpose Solution for Contact Lenses with a Sucralose

| Ingredient | % w/w | |
|---|---|---|
| Sucralose | 1.00 | |
| Tetronic 1107 | 1.00 | |
| Sodium Borate | 0.09 | |
| Boric Acid | 0.64 | |
| EDTA | 0.11 | |
| PHMB | 1.0 | ppm |
| Dequest 2016 | 0.03 | |
| Sodium Chloride | 0.49 | |
| Purified Water | Q.S. to 100 | gm |

### Example 6

### Flavored Multi-Purpose Solution for Contact Lenses

A multi-purpose solution for contact lenses with cocoa flavor was prepared with the following formulation shown below in Table 8. Cocoa flavor was added by mixing the indicated amount with prepared Multi-purpose solution for contact lenses.

TABLE 8
Multi-Purpose Solution for Contact Lenses with Cocoa Flavor

| Ingredient | % w/w | |
|---|---|---|
| Sucralose | 1.00 | |
| Tetronic 1107 | 1.00 | |
| Sodium Borate | 0.09 | |
| Citric Acid | 0.30 | |
| Cocoa Flavor | 0.50 | |
| EDTA | 0.11 | |
| PHMB | 1.0 | ppm |
| Dequest 2016 | 0.03 | |
| Sodium Chloride | 0.49 | |
| Purified Water | Q.S. to 100 | gm |

### Example 7

### Multipurpose Lens Care Solutions Using PHMB (ReNu MultiPlus®) and Alexidine as Disinfectants with a Sweetener

Multi-purpose solutions for contact lenses with sucralose were prepared with following the formulation, shown in Table 9. Both solutions provide sustained comfort yet clean, disinfect, rinse, store, and remove protein daily for soft contact lenses without the need to rub. Sucralose was added by mixing the indicated amount.

TABLE 9
Multipurpose Lens Care Solutions using PHMB (ReNu MultiPlus ®)
and Alexidine as Disinfectants with a Sucralose

| | PHMB-Based | | Alexidine-Based | |
|---|---|---|---|---|
| Ingredient | (% w/w) | | (% w/w) | |
| Pluronic P123 | — | | 2.00 | |
| Tetronic 1107 | 1.00 | | 1.00 | |
| Sodium Chloride | 0.49 | | 0.09 | |
| Boric Acid | 0.64 | | 0.85 | |
| Sodium Borate | 0.09 | | — | |
| EDTA | 0.11 | | — | |
| Sodium Phosphate | — | | 0.15 | |
| (monobasic) | | | | |
| Sodium Phosphate | — | | 0.31 | |
| (Dibasic) | | | | |
| Polymer JR | — | | 0.02 | |
| PHMB HCl | 1.1 | ppm | — | |
| Alexidine 2HCl | — | | 4.5 | ppm |
| Sucralose | 1.00 | | 1.00 | |
| Dequest 2016 | 0.10 | | 0.10 | |
| Purified Water | Q.S. to 100 | gm | Q.S to 100 | gm |

### Example 8

### Multipurpose Lens Care Solutions using PHMB (ReNu MultiPlus®) and Alexidine as Disinfectants with a Sweetener and a Flavorant

Multi-purpose solutions for contact lenses with watermelon flavor including sucralose as a sweetener were prepared with following formulation, shown in Table 10. The watermelon flavor was added by mixing the indicated amount.

TABLE 10
Multi-Purpose Solutions for Contact Lenses with Watermelon Flavor
including Sucralose

| | | | Alexidine- | |
|---|---|---|---|---|
| | PHMB-Based | | Based | |
| Ingredient | (% w/w) | | (% w/w) | |
| Pluronic P123 | — | | 2.00 | |
| Tetronic 1107 | 1.00 | | 1.00 | |
| Sodium Chloride | 0.49 | | 0.09 | |
| Boric Acid | 0.64 | | 0.85 | |
| Sodium Borate | 0.09 | | — | |
| Boric Acid | 0.64 | | 0.85 | |
| Sodium Borate | 0.09 | | — | |
| EDTA | 0.11 | | — | |
| Sodium Phosphate | — | | 0.15 | |
| (Monobasic) | | | | |
| Sodium Phosphate | — | | 0.31 | |
| (dibasic) | | | | |
| Polymer JR | — | | 0.02 | |
| PHMB HCl | 1.1 | ppm | — | |
| Alexidine 2HCl | — | | 4.5 | ppm |
| Sucralose | 1.00 | | 1.00 | |
| Watermelon Flavor | 0.40 | | 0.40 | |
| Dequest 2016 | 0.10 | | 0.10 | |
| Purified Water | Q.S. to 100 | gm | Q.S. to 100 | gm |

The claimed invention is:

1. An ophthalmic solution comprising at least one flavoring agent, wherein the flavoring agent is present in an amount effective to mask the inherent taste of the ophthalmic solution or to add flavors to the ophthalmic solution.

2. The ophthalmic solution of claim 1, wherein the flavoring agent is a sweetener.

3. The ophthalmic solution of claim 2, further comprising a flavoring agent selected from the group consisting of a sour flavoring agent, a bitter flavoring agent, and mixtures thereof.

4. The ophthalmic solution of claim 2, wherein the flavoring agent is present in an amount from about 0.0001 to 20 weight percent.

5. The ophthalmic solution of claim 4, wherein the flavoring agent is present in an amount from about 0.1 to 5 weight percent.

6. The ophthalmic solution of claim 2, wherein the ophthalmic solution is selected from a contact lens solution, an eye drop formulation, and a pharmaceutical composition containing at least one active pharmaceutical ingredient for the treatment of an eye disease.

7. The ophthalmic solution of claim 6, wherein the ophthalmic solution is a contact lens solution selected from a cleaning solution, a wetting solution, a soaking solution, a conditioning solution, and a storing solution.

8. The ophthalmic solution of claim 6, wherein the ophthalmic solution is an eye drop formulation.

9. The ophthalmic solution of claim 8, wherein the eye drop formulation is selected from a solution to soothe eye irritation, a moisturizing solution, a contact lens rewetting solution, and a contact lens lubricating solution.

10. The ophthalmic solution of claim 6, wherein the ophthalmic solution is a pharmaceutical composition for the treatment of an eye disease and the active pharmaceutical ingredient is selected from an anti-inflammatory agent, an ophthalmic beta blocker, and a high potency antioxidant and mineral supplement.

11. The ophthalmic solution of claim 2, wherein the sweetener is a natural sugar or a sugar substitute of artificial origin.

12. The ophthalmic solution of claim 11, wherein the sweetener is sucrose.

13. The ophthalmic solution of claim 12, wherein the sweetener is selected from the group consisting of sucralose, isomalt, aspartame, saccharin, lactitol, or high-fructose corn syrup.

14. The ophthalmic solution of claim 13, wherein the sweetener is sucralose.

15. The ophthalmic solution of claim 12, wherein the sweetener is selected from the group consisting of sorbitol, mannitol, xylitol, and maltitol.

16. The ophthalmic solution of claim 1, wherein the flavoring agent is selected from the group consisting of natural flavors, natural fruit flavors, artificial flavors, artificial fruit flavors, flavor enhancers and mixtures thereof.

17. The ophthalmic solution of claim 16, further comprising a flavoring agent selected from the group consisting of a sweetener, a sour flavoring agent, a bitter flavoring agent, and mixtures thereof.

18. The ophthalmic solution of claim 16, wherein the flavoring agent is present in an amount from about 0.0001 to 20 weight percent.

19. The ophthalmic solution of claim 18, wherein the flavoring agent is present in an amount from about 0.1 to 5 weight percent.

20. The ophthalmic solution of claim 16, wherein the flavoring agent is an orange flavor.

21. The ophthalmic solution of claim 16, wherein the flavoring agent is a lemon flavor.

22. The ophthalmic solution of claim 16, wherein the flavoring agent is a cocoa flavor.

23. The ophthalmic solution of claim 16, wherein the flavoring agent is a watermelon flavor.

24. The solution of claim 16, wherein the flavoring agent is selected from the group consisting of 6-Methyl Coumarin, Anethole USP, Cassia Oil, Cassia Oil Redistilled, Cinnamon Bark Oil, Cinnamon Leaf Oil Cleaned, Clove Bud Oil English Distilled SAS, Clove Leaf Oil Redistilled, Cocal™, Cocoa Distillate (Nat.), Cocoa Essence Dark, Cocoa Essence White, Coriander Oil, delta Decalactone, Dimethyl Benzyl Carbinyl Butyrate, Ethyl-2-Methyl Butyrate, Ethyl-3-Hydroxy Butyrate, Ethyl Butyrate, Ethyl Iso Butyrate, Ethyl Iso Valerate, Ethyl Oxanoate 369, Eucalyptus Oil 80%, Farnesene 1% PG/ETOH, Furfurrole 302, gamma-Decalactone, gamma-Hexalactone, gamma-Octalactone, gamma Dodecalactone, Ginger Oil Chinese, Ginger Oil Nigerian English Distilled SAS, Grapefruit Key, Heptan-2-One (Nat.), Hexene-3-One4, Hexyl Acetate, Homo Cyclocitral, beta, Honey Distillate Nat., Ionone Beta, Iso Amyl Iso Valerate, Iso Butyl Caproate, Iso Butyl Furyl Propionate, Iso Fragarone-030, Iso Fragarone, 1% ETOH™, Juniperberry Oil English Distilled SAS, Ketone Mix, Kumarone™, Lemon Oil 5× Sas, Lemon Oil Terpeneless Sas, Lemonless Lemon Key, Lime Oil Terpeneless, Linalool 75/80% Ex Orange (Nat.), Linalyl Acetate (Nat.), Mangone 5% ETOH™, Methional, Methyl Butyric Acid (2), Methyl Ketones (Nat.), Methyl Oxycyclosulfide 719, Mushroom Extract, Natural Flavor (99% Vanillin), Nat. Cocoa Butter Distillate, Nonan-2-One (Nat.), Octanal 35% (Nat.), Octen-4-one-2, Olibanum Oil English Distilled SAS, Orange Oil 15× Decolorized M3706, Orange Oil 950 (10×), Orange Oil Terpeneless 2501, Oxaromate-884, Oxycyclothione-030, Paradiff™ 0.01% ETOHGR, Paradiff™ 0.01% Grapefruit Oil, Peach Flavor Key, Peppermint Oil Redistilled Yakima, Peppermint Oil Spec. Fractions, Phenyl Ethyl 2-Methyl Butyrate, Phenyl Ethyl Acetate, Phenyl Ethyl Alcohol, Phenyl Oxaromate-681, Pimento Berry Oil English Distilled SAS, Pimento Leaf Oil, Pimento Leaf Oil Cleaned, Pineapple Compound 15% ETOH GR, Pineapple Compound 15% PG, Popcorn Chemical, Propionic Acid, Raspberry Flavor Key, Robustone 1.0% ETOH™, Robustone™, Sclareolide, Sesame Distillate Nat., Sinensals (Nat.), Starter Distillate 15× W/S, Strawberriff, Strawberry Base, Strawberry Flavor Key, Strawberry Flavor Key, Succinic Acid, Sulfurome-015, Sweetness Modifier, Tetrahydro Terrazine-014™, Thionol-935, Thionol-966, trans-2-Hexenal, Trimenal Acetate 399 1% ETOH™, Tropical Fruit Key Base, Tropical Fruit Key Base, Undecan-2-One (Nat.), Varamol- 106 10% ETOH, Varamol-106 10% NEBM5, and Varamol-106 10% PG, and mixtures thereof.
